(19) United States
(12) Patent Application Publication
(10) Pub. No.: US 2026/0054000 A1
(43) Pub. Date: Feb. 26, 2026

(54) EAR IRRIGATION AND SUCTION DEVICE

(71) Applicant: CleanEar Inc., Odessa, TX (US)

(72) Inventors: Christopher B. Perry, Toledo, OH (US); Tito Vasquez, Findlay, OH (US); Puthalath Raghuprasad, Odessa, TX (US)

(73) Assignee: CleanEar Inc., Odessa, TX (US)

(21) Appl. No.: 19/309,153

(22) Filed: Aug. 25, 2025

Related U.S. Application Data
(60) Provisional application No. 63/686,975, filed on Aug. 26, 2024.

(51) Int. Cl. A61M 1/00

(52) U.S. Cl. CPC A61M 1/77; A61M 2210/0662

(57) ABSTRACT

An ear irrigation and suction device, and methods of using the same, are disclosed. The irrigation and suction device includes a fluid reservoir, a vacuum pump, and a detachable fluid appendage. The fluid appendage includes a primary channel that delivers a negative pressure to remove debris from an ear canal through suction and a secondary channel that supplies fluid under positive pressure to the ear canal for effective irrigation. The device may also include a controller for managing various operational modes, allowing for customized ear irrigation and suction tailored to individual needs.

### RELATED APPLICATIONS

This application claims priority to U.S. Provisional Application No. 63/686,975 filed under 35 U.S.C. § 111 (b) on Aug. 26, 2024, the disclosure of which is incorporated herein by reference in its entirety for all purposes.

### BACKGROUND

Ear irrigation is a common medical procedure used to remove earwax (cerumen) and other debris from the ear canal. Excessive earwax buildup can cause various issues, including hearing loss, pain, and infections. Traditional methods of ear irrigation involve the manual administration of fluid into the ear canal, often using syringes or other rudimentary tools. However, these methods can be inefficient, uncomfortable, and sometimes ineffective, especially when dealing with stubborn or impacted earwax.

In recent years, there has been a growing demand for more advanced and efficient ear irrigation devices that can provide a more controlled and effective cleaning process. Such devices aim to improve the user experience by offering features such as automated fluid delivery and suction, ergonomic designs, and customizable operational modes. The development of an ear irrigation and suction device that integrates these advanced features can significantly enhance the safety, comfort, and effectiveness of ear cleaning procedures.

### SUMMARY

Provided is an irrigation and suction device for an ear comprising a fluid reservoir; a vacuum pump; and a fluid appendage having a primary channel therein and a secondary channel therein, wherein the primary channel is in fluid communication with the vacuum pump and the secondary channel is in fluid communication with the fluid reservoir, the fluid appendage further comprising a main tip where the primary channel terminates and a nozzle where the secondary channel terminates, wherein the nozzle extends longitudinally outward beyond the main tip.

In certain embodiments, the secondary channel includes a dogleg lumen portion, a concentric lumen portion, and a top lumen portion. In particular embodiments, the primary channel longitudinally defines a primary channel axis, the dogleg lumen portion is on a first side of the primary channel axis, and the top lumen portion is on a second side of the longitudinal axis. In particular embodiments, the top lumen portion is on the second side of the primary channel axis. In particular embodiments, the concentric lumen portion is concentric with the primary channel in a middle portion of the fluid appendage.

In certain embodiments, the nozzle includes a non-circular opening. In particular embodiments, the nozzle has a perimeter which includes a flat portion and a curved portion, where the curved portion is closer to the primary channel axis than the flat portion is. In certain embodiments, the main tip is curved.

Further provided is an irrigation and suction device for an ear comprising a fluid reservoir; a vacuum pump; and a fluid appendage having a primary channel therein and a secondary channel therein, wherein the primary channel is in fluid communication with the vacuum pump and the secondary channel is in fluid communication with the fluid reservoir; wherein a portion of the secondary channel wraps around a portion of the primary channel within the fluid appendage. In certain embodiments, the portion is a concentric lumen portion which is concentric with the primary channel in a middle portion of the fluid appendage. In certain embodiments, the primary channel longitudinally defines a primary channel axis, the secondary channel includes a dogleg lumen portion on a first side of the primary channel axis, and the fluid appendage further comprises a nozzle in fluid communication with the secondary channel on an opposing second side of the primary channel axis. In particular embodiments, the nozzle includes a non-circular opening. In particular embodiments, the nozzle has a perimeter which includes a flat portion and a curved portion, where the curved portion is closer to the primary channel axis than the flat portion is.

Further provided is an irrigation and suction device for an ear comprising a fluid reservoir; a vacuum pump; and a fluid appendage having a primary channel therein and a secondary channel therein, wherein the primary channel is in fluid communication with the vacuum pump and the secondary channel is in fluid communication with the fluid reservoir, the primary channel longitudinally defining a primary channel axis; wherein the secondary channel is on a first side of the primary channel axis at a proximal end of the fluid appendage, and the secondary channel is on a second side of the primary channel axis at a distal end of the fluid appendage. In certain embodiments, the secondary channel includes a concentric lumen portion which is concentric to the primary channel in a middle portion of the fluid appendage. In particular embodiments, the secondary channel includes a dogleg lumen portion between the proximal end and the concentric lumen portion. In certain embodiments, the secondary channel wraps around a portion of the primary channel. In certain embodiments, the secondary channel terminates at a nozzle, and the primary channel terminates at a main tip, wherein the nozzle extends longitudinally beyond the main tip. In particular embodiments, the nozzle includes a non-circular opening. In particular embodiments, the nozzle has a perimeter which includes a flat portion and a curved portion.

Further provided is an irrigation and suction device for an ear comprising a fluid reservoir; a vacuum pump; and a fluid appendage having a primary channel therein and a secondary channel therein, wherein the primary channel is in fluid communication with the vacuum pump and the secondary channel is in fluid communication with the fluid reservoir; wherein the secondary channel includes a dogleg lumen portion with a curve. In certain embodiments, the secondary channel further includes a concentric lumen portion which is concentric with the primary channel in a middle portion of the fluid appendage. In certain embodiments, the secondary channel wraps around a portion of the primary channel.

In any embodiment of the irrigation and suction device, the irrigation and suction device may further comprise a controller in signal connection to at least one of the fluid reservoir and the vacuum pump; wherein the controller is configured to actuate the at least one of the fluid reservoir and the vacuum pump between an active state and a deactivated state.

In any embodiment of the irrigation and suction device, the fluid appendage may be removably attached to a component of the irrigation and suction device.

Further provided is a fluid appendage for an ear irrigation and suction device, the fluid appendage comprising a primary channel terminating at a main tip and a secondary channel terminating at a nozzle, wherein either (i) the secondary channel wraps around a portion of the primary channel, (ii) the nozzle extends a distance outward from the main tip, or (iii) the secondary channel includes a dogleg lumen portion having a curve.

Further provided is a method of irrigating and suctioning an ear, the method comprising inserting a main tip and a nozzle of a fluid appendage of an irrigation and suction device into an ear canal; applying negative pressure to the ear canal through a primary channel in the fluid appendage; and pumping a fluid from a fluid reservoir into the ear canal through a secondary channel in the fluid appendage; wherein the nozzle extends farther into the ear canal than the main tip.

Further provided is a method of irrigating and suctioning an ear, the method comprising inserting a main tip and a nozzle of a fluid appendage of an irrigation and suction device into an ear canal; applying negative pressure to the ear canal through a primary channel in the fluid appendage; and pumping a fluid from a fluid reservoir into the ear canal through a secondary channel in the fluid appendage; wherein fluid is delivered to the ear canal through a non-circular opening in the nozzle.

Further provided is a method of irrigating and suctioning an ear, the method comprising inserting a main tip and a nozzle of a fluid appendage of an irrigation and suction device into an ear canal; applying negative pressure to the ear canal through a primary channel in the fluid appendage; and pumping a fluid from a fluid reservoir into the ear canal through a secondary channel in the fluid appendage; wherein the secondary channel wraps around, and is concentric with, a portion of a primary channel within the fluid appendage.

Further provided is a method of irrigating and suctioning an ear, the method comprising inserting a main tip and a nozzle of a fluid appendage of an irrigation and suction device into an ear canal of a dog; applying negative pressure to the ear canal through a primary channel in the fluid appendage; and pumping a fluid from a fluid reservoir into the ear canal through a secondary channel in the fluid appendage; wherein the primary channel terminates at a curved main tip, the secondary channel terminates at a nozzle, and the curved main tip extends farther into the ear canal than the nozzle.

Further described is an irrigation and suction device which includes an irrigation and suction unit and a fluid appendage. The irrigation and suction unit defines a gripping body and a unit head. The irrigation and suction unit includes a fluid reservoir and a vacuum pump. Both the fluid reservoir and vacuum pump are disposed within the gripping body. The fluid appendage is configured to be attached to the unit head. The fluid appendage is in fluid communication with at least one of the fluid reservoir and the vacuum pump. The fluid appendage includes a primary channel and a secondary channel. The primary channel is configured to apply a negative pressure supplied by the vacuum pump to the ear canal. The primary channel defines a primary channel plane and a primary channel lumen. The secondary channel is configured to supply fluid under positive pressure from the fluid reservoir into the ear canal. The secondary channel is partially disposed within the primary channel lumen. The secondary channel is disposed on an opposite side of the primary channel plane than the primary channel such that the secondary channel protrudes into the ear canal further than the primary channel when in use.

Further described is an irrigation and suction device which includes a fluid reservoir and a vacuum pump. Both the fluid reservoir and vacuum pump are disposed within a gripping body. The fluid appendage is configured to be attached to a unit head. The fluid appendage is in fluid communication with at least one of the fluid reservoir and the vacuum pump. The fluid appendage includes a primary channel and a secondary channel. The primary channel is configured to apply a negative pressure supplied by the vacuum pump to an ear canal of a subject, who may be a human or animal such as a dog or cat. The primary channel defines a primary channel plane and a primary channel lumen. The secondary channel is configured to supply fluid under positive pressure from the fluid reservoir into the ear canal. The secondary channel extends from a first side of the primary channel plane to a second side of the primary channel plane, wrapping around the primary channel in part, and the secondary channel is configured to protrude further into the ear canal than the primary channel.

Further provided is an irrigation and suction device for an ear comprising a fluid reservoir; a vacuum pump; and an animal ear fluid appendage having a primary channel therein and a secondary channel therein, wherein the primary channel is in fluid communication with the vacuum pump and the secondary channel is in fluid communication with the fluid reservoir, the animal ear fluid appendage having an elongate tubular member extending from an elongate tubular member proximal end at a main tip to an elongate tubular member distal end, wherein the elongate tubular member has a bend in an elongate tubular member intermediate portion.

In certain embodiments, the elongate tubular member distal end defines a flange, the flange having a larger circumference than the elongate tubular member intermediate portion. In particular embodiments, the flange defines a plurality of crescent-shaped nozzles in fluid communication with the secondary channel.

In certain embodiments, the elongate tubular member proximal end fits over the main tip.

In certain embodiments, the secondary channel terminates at a nozzle, wherein the nozzle extends longitudinally outward beyond the elongated tubular member distal end.

In certain embodiments, the irrigation and suction device further comprises a seal between the main tip and the elongate tubular member proximal end.

In certain embodiments, the bend is at an angle of greater than 90 degrees relative to a longitudinal axis of the elongate tubular member proximal end.

Further provided is a fluid appendage for an irrigation and suction device comprising a main tip comprising a first inlet providing access to a primary channel and a second inlet providing access to a secondary channel; an elongate tubular member extending from an elongate tubular member proximal end at the main tip to an elongate tubular member distal end with an elongate tubular member intermediate portion therebetween, wherein the elongate tubular member is connected to the main tip; and a bend in the elongate tubular member intermediate portion; wherein the elongate tubular member is configured to allow passage of a negative pressure through the primary channel to the elongate tubular member distal end, and to allow passage of a fluid through the secondary channel to the elongate tubular member distal end; and wherein the primary channel is concentric with the secondary channel in the elongate tubular member intermediate portion.

In certain embodiments, the secondary channel terminates at a nozzle on the distal end, the nozzle extending longitudinally beyond the distal end.

In certain embodiments, the fluid appendage further comprises an elongate tubular member protrusion housing a nozzle which fluidly connects the secondary channel to an elongate tubular member perimeter channel within the elongate tubular member intermediate portion.

In certain embodiments, the primary channel is concentric with the secondary channel from the elongate tubular member proximal end to the elongate tubular member distal end.

In certain embodiments, the bend is at an angle of greater than 90 degrees relative to a longitudinal axis of the elongate tubular member proximal end.

Further provided is a method of irrigating and suctioning an ear, the method comprising inserting the main tip and the nozzle of the fluid appendage of the irrigation and suction device described herein into an ear canal of a subject; applying negative pressure to the ear canal through the primary channel in the fluid appendage; and pumping a fluid from the fluid reservoir into the ear canal through the secondary channel in the fluid appendage; wherein the nozzle extends farther into the ear canal than the main tip.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an irrigation and suction device having a fluid appendage in accordance with the present disclosure.

FIG. 2 illustrates an exploded view of a non-limiting example irrigation and suction device having a fluid appendage in accordance with the present disclosure.

FIG. 3 illustrates a perspective view of a fluid appendage in accordance with the present disclosure.

FIG. 4 illustrates a perspective fluid of the fluid appendage depicted in FIG. 3.

FIG. 5 illustrates another perspective view of a fluid appendage in accordance with the present disclosure, where the fluid appendage includes appendage protrusions.

FIG. 6 illustrates a front view of a fluid appendage in accordance with the present disclosure.

FIG. 7 illustrates a perspective view of a fluid appendage in accordance with the present disclosure.

FIG. 8 illustrates a flowchart of a non-limiting example irrigation and suction method.

FIG. 9 illustrates a perspective view of an animal ear fluid appendage in accordance with the present disclosure.

FIG. 10 illustrates a perspective view of another animal ear fluid appendage in accordance with the present disclosure.

FIG. 11 illustrates a sectional view of the animal ear fluid appendage illustrated in FIG. 10.

FIG. 12 illustrates a perspective view of the animal ear fluid appendage illustrated in FIG. 10.

FIG. 13 illustrates a partial perspective view of the animal ear fluid appendage illustrated in FIG. 10.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

The present disclosure addresses the needs in the art by providing an irrigation and suction device with a sophisticated design that includes a specialized fluid appendage configured to provide easy and convenient removal of debris from an ear canal. The device described herein offers a reliable and user-friendly solution for ear irrigation and suction, making the process more efficient and less cumbersome for both medical professionals and patients.

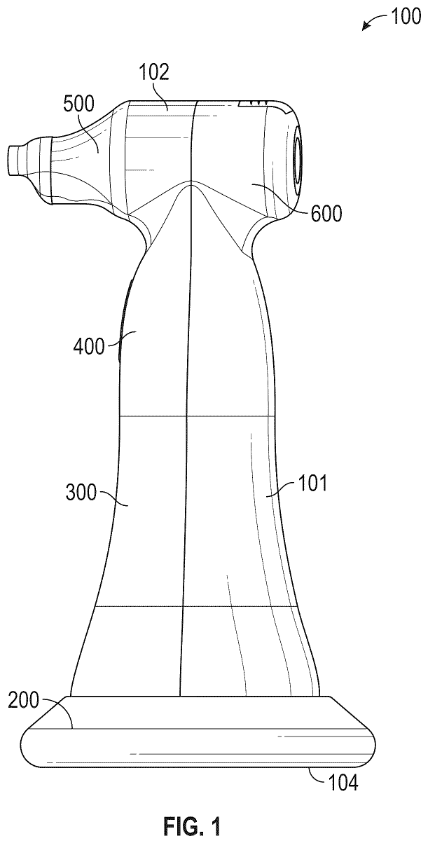

FIG. 1 illustrates an irrigation and suction device 100. The irrigation and suction device 100 may be used to irrigate and suction an ear of a subject, who may be a human or an animal such as a dog or cat. The irrigation and suction device 100 may be configured to input fluid into an ear using a positive pressure. The fluid be used to dislodge, dissolve, and/or expel debris from an ear such as, but not limited to, cerumen. Additionally, the irrigation and suction device 100 may be configured to remove fluid or debris, such as cerumen, from the ear using a negative pressure.

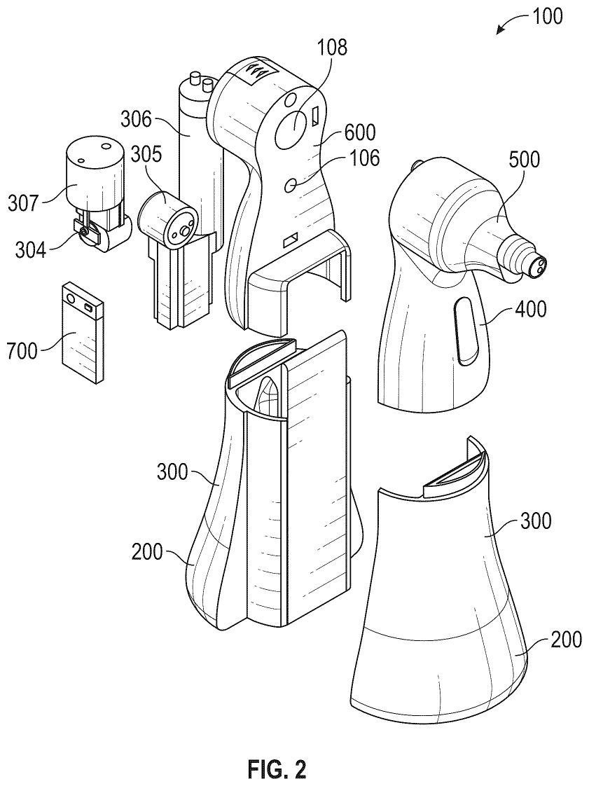

Referring to FIGS. 1-2, the irrigation and suction device 100 includes a body 101 having a working end 102 and a base end 104. The working end 102 is the end of the irrigation and suction device 100 configured to be grasped by a user for irrigating and suctioning an ear. The base end 104 is the end of the irrigation and suction device 100 which is configured to supply fluid and/or suction to the ear. The base end 104 includes a base 200 and a gripping body 300. The working end 102 includes a unit head 600 and a fluid appendage 500. A middle housing 400 is disposed between the components at the base end 104 and the components at the working end 102. Thus, the irrigation and suction device 100 may include a body 101, a base 200, a gripping body 300, a middle housing 400, a fluid appendage 500, and a unit head 600. The base 200 is at the base end 104 of the irrigation and suction device 100, while the fluid appendage 500 and unit head 600 are disposed at the working end 104 of the irrigation and suction device 100. As such, the fluid appendage 500 and unit head 600 are disposed opposite the base 200. The gripping body 300 may be disposed between the base 200 and the fluid appendage 500. Some embodiments may also include a fluid reservoir 306, a fluid pump 305, a vacuum pump 304, a repository 307, an internal battery 700, and/or a controller (not illustrated), some or all of which may be housed within the unit head 600, the middle housing 400, and/or the base 200. The fluid reservoir 306 may further include a separate fluid pump 305 for pumping fluid out of the fluid reservoir 306 on command by the controller or any other suitable means. In embodiments having a controller, the controller may be configured to employ a plurality of operational modes such as, but not limited to, low, medium and high suction. All of the components of the irrigation and suction device 100 may be constructed from materials suitable for medical devices such as, but not limited to, medical-grade plastics, stainless steel, acrylonitrile butadiene styrene (ABS) filament, thermoplastic polyurethane (TPU) filament, or any other suitable materials.

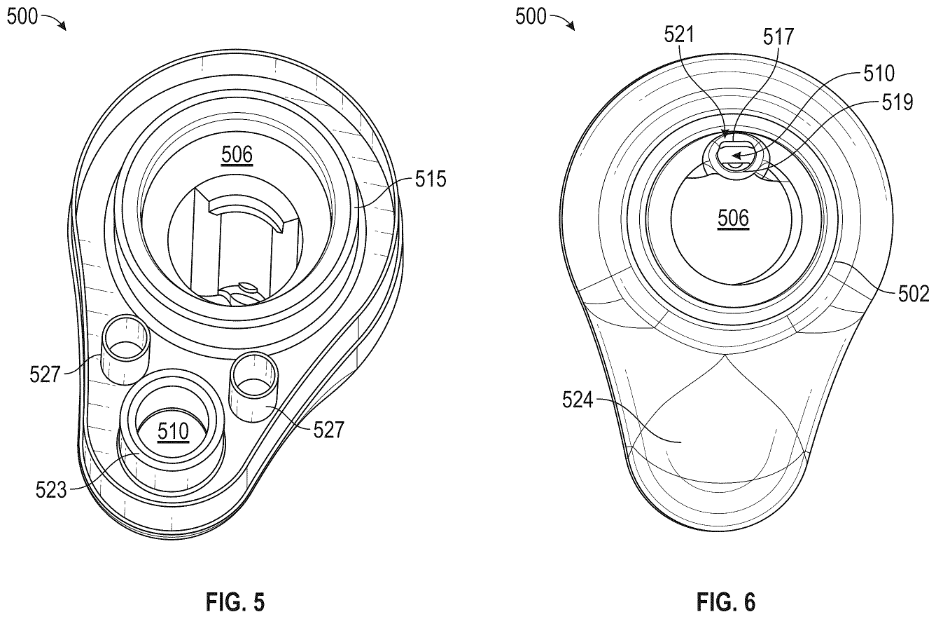

Referring to FIG. 2, the irrigation and suction device 100 may include a plurality of flow channels 106, 108 through the unit head 600. In some embodiments, the irrigation and suction device 100 may include both a clean fluid channel 106 and a debris fluid channel 108. The internal channels 106, 108 may be constructed with smooth surfaces and gradual bends to enhance fluid dynamics and reduce turbulence. As will be described later and with reference to FIGS. 4-5, the clean fluid channel 106 is configured to be fluidly attached to a secondary channel 506 within the fluid appendage 500, and the debris fluid channel 108 is configured to be fluidly attached to a primary channel 506 within the fluid appendage 500. The clean fluid channel 106 is configured to facilitate the input of clean fluid into the ear, via passage of the clean fluid through the secondary channel 510 in the fluid appendage 500. The debris fluid channel 108 is configured to facilitate the removal of fluid or debris to be removed from the ear, via passage of the fluid or debris through the primary channel 506 within the fluid appendage 500.

Referring still to FIG. 2, in some embodiments, at least one of the plurality of flow channels 106, 108 may be configured to flow outside of the irrigation and suction device 100. For example, the irrigation and suction device 100 may be configured to receive fluid from an outside source into the irrigation and suction device 100. Additionally, or alternatively, the irrigation and suction device 100 may be configured to deposit debris fluid removed from an ear to an outside repository or location (i.e., external to the irrigation and suction device 100). In other embodiments, the plurality of flow channels 106, 108 may be configured to flow within the irrigation and suction device 100. For example, the irrigation and suction device 100 may be configured to pull clean fluid from a fluid reservoir 306 within the irrigation and suction device 100, input the clean fluid into the ear such that the clean fluid mixes with debris within the ear, and then suction debris fluid out of the ear into a repository 307 disposed within the irrigation and suction device 100. It is understood, however, that the repository 307 need not be contained within the irrigation and suction device 100, and in fact the suctioned debris fluid may be directed out of the irrigation and suction device 100.

Referring to FIGS. 1-2, the base 200 provides structural support for the irrigation and suction device 100. Furthermore, the base 200 may be used to house portions of the plurality of fluid channels 106, 108. In some embodiments, the base 200 serves as the primary interface for fluid ingress into the irrigation and suction device 100 and fluid egress out of the irrigation and suction device 100, although this is not strictly necessary. The base 200 may be constructed from a durable, medical-grade material. The base 200 may be configured to include multiple channels 106, 108 that facilitate fluid flow. In some embodiments, the base 200 may include a base inlet and a base outlet (not illustrated), where the base inlet is configured to facilitate the transmission of clean fluid into the irrigation and suction device 100 and the base outlet is configured to facilitate the removal of debris-filled fluid from the irrigation and suction device 100. The base inlet may be connected to an external fluid source or to a fluid reservoir 306 housed within the gripping body 300. The base inlet may feature a secure connection, such as a threaded or quick-connect fitting, to provide a leak-proof interface. This connection allows fluid to flow from the external source into the irrigation and suction device 100, preparing the irrigation and suction device 100 for the irrigation and suction process. The base outlet ensures that the fluid, along with any dislodged debris from the ear, expelled from an ear canal may exit the irrigation and suction device 100 when desired. The base outlet may include a secure connection to provide a leak-free fluid pathway. The base outlet may be connected to the vacuum pump 304 or another extraction system within or external to the irrigation and suction device 100, enabling controlled removal of the debris and the fluid. Within the base 200, internal channels may be precisely engineered to guide fluid flow between the base inlet, fluid reservoir 306, and base outlet. These internal channels are configured to minimize resistance and prevent blockages, ensuring smooth and consistent fluid movement.

The base 200 is configured to integrate seamlessly with other components of the irrigation and suction device 100, such as the gripping body 300, fluid reservoir 306, and the middle housing 400. The base 200 provides a stable foundation for these components, ensuring that the irrigation and suction device 100 operates as a cohesive unit. The base 200 may include alignment features or mounting points that facilitate secure attachment to the gripping body 300, enhancing the overall stability and integrity of the irrigation and suction device 100. In some embodiments, the base 200 may include additional features such as fluid filters or pressure regulators (not illustrated). These components may be used to ensure that only clean, properly pressurized fluid enters the ear canal. The base 200 may also be configured to accommodate different sizes or types of fluid reservoirs 306.

Referring still to FIG. 2, the gripping body 300 may house important functional components of the irrigation and suction device 100. The gripping body 300 is an elongated structure. The gripping body 300 may include an external gripping surface which may incorporate a textured surface or finger indentations to enhance grip. Such structures may be used to prevent slippage during use, especially when handling fluids. The gripping body 300 may be configured to house a plurality of pumps and reservoirs. For example, the gripping body 300 may be configured to house the fluid reservoir 306 and the vacuum pump 304. Additionally, the gripping body 300 may include internal channels and connections that facilitate fluid communication between the fluid reservoir 306, vacuum pump 304, and fluid appendage 500. The gripping body 300 may also include seals and gaskets at connection points to maintain a fluid-tight system. Externally, the gripping body 300 may be configured to interface seamlessly with the middle housing 400 and fluid appendage 500. The upper section of the gripping body 300 may terminate in a connector or coupling that allows for the secure attachment of the middle housing 400. This connection may ensure that the fluid appendage 500, when attached, is in direct fluid communication with at least one of the fluid reservoir 306 and vacuum pump 304. In some embodiments, the gripping body 300 may be modular, allowing for the attachment of various accessory components or different types of fluid appendages.

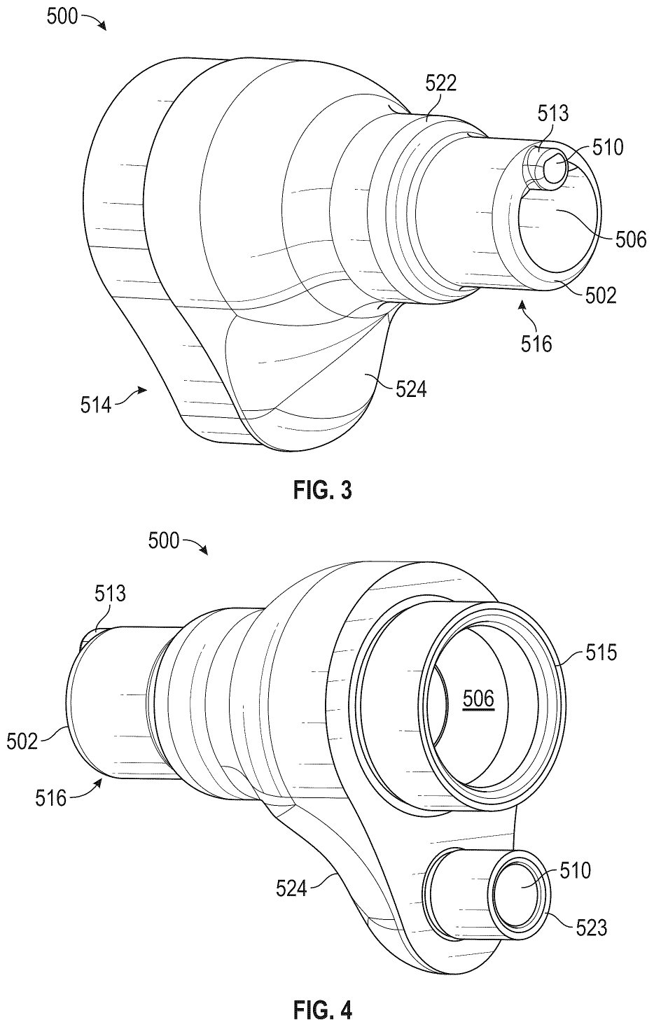

Referring still to FIG. 2, a fluid reservoir 306 may be housed within the gripping body 300 of the irrigation and suction device 100. The fluid reservoir 306 may be configured to hold a predetermined volume of fluid. The fluid reservoir 306 may be constructed from a durable, medical-grade material that is resistant to degradation from common ear irrigation fluids, such as saline solutions or specialized ear cleaning solutions. The fluid reservoir 306 may be configured to be in fluid communication with the secondary channel 510 of the fluid appendage 500, depicted in FIG. 3. This connection may be achieved through internal channels 106, 108 or tubing that runs from the fluid reservoir 306 to the fluid appendage 500. To facilitate the filling and refilling of the fluid reservoir 306, the fluid reservoir 306 may be equipped with a fill port. The fill port is configured to be securely scaled to prevent leaks. The fill port may incorporate a one-way valve or a screw-cap to allow fluid to be added without the risk of spillage or contamination. In some embodiments, the fluid reservoir 306 may be removable from the gripping body 300, such that a user may clean and refill the fluid reservoir 306. In some embodiments, a fluid level sensor may be integrated within the fluid reservoir 306 to monitor the fluid level and provide feedback to a controller or a gauge. The fluid level sensor ensures that the user is alerted when the fluid level is low. The fluid reservoir 306 may also include internal baffling or compartmentalization (not illustrated), to prevent fluid sloshing during use. The baffling may ensure that the fluid remains evenly distributed within the fluid reservoir 306, maintaining consistent fluid pressure and flow. This can be accomplished by any suitable means such as, for example, a plurality of lights on the body 101 indicating low, medium, and high fluid levels or a gauge on the body 101. The fluid reservoir 306 may be connected to a fluid pump 305 housed within the gripping body 300. The fluid pump 305, when actuated by the user, applies positive pressure to the fluid within the fluid reservoir 306, forcing the fluid through the internal channel 106 and into the secondary channel 510 of the fluid appendage 500.

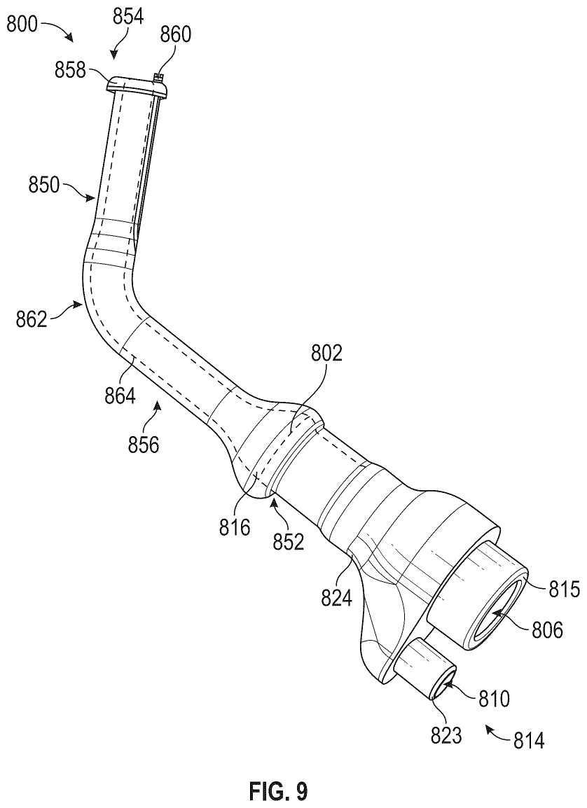

Referring to FIGS. 1-2, the middle housing 400 is disposed between the gripping body 300 and the fluid appendage 500. The middle housing 400 may be configured to include an appendage interface that allows for the attachment of various tips such as, but not limited to, the fluid appendage 500 or an animal ear fluid appendage 800 illustrated in FIG. 9, to facilitate the delivery and removal of fluids during the irrigation and suction process. The middle housing 400 may define an attachment, such as a threaded connection, snap-fit, or bayonet mount. This attachment ensures that the fluid appendage 500 remains firmly in place during use, preventing leaks and ensuring the efficient delivery and removal of fluids. The middle housing 400 may also include a plurality of channels that are in fluid communication with both the fluid reservoir 306 and the vacuum pump 304 housed within the gripping body 300. However, the internal channels 106, 108 do not need to run through the middle housing 400, as they can extend from the fluid reservoir 306 and vacuum pump 304 through the unit head 600 to the fluid appendage 500, without being within the middle housing 400. The middle housing 400 may also feature integrated controls or sensors which interact with a controller. For example, the middle housing 400 may include pressure sensors to monitor the fluid pressure within the channels 106, 108, providing feedback to the controller to adjust the operation of the fluid reservoir 306 and vacuum pump 304 as needed. The sensors may be used to ensure that fluid delivery and suction are maintained at predetermined levels.

Referring to FIG. 2, the fluid pump 305 is configured to supply fluid under positive pressure from the fluid reservoir 306 into the ear canal. The fluid pump 305 may be used in facilitating the irrigation process by dislodging, dissolving, and expelling debris from the ear. The fluid pump 305 may be housed within the gripping body 300 of the irrigation and suction device 100. The fluid pump 305 may be used to generate positive pressure to supply fluid from the fluid reservoir 306 to the ear canal. The fluid pump 305 is in fluid communication with the internal channel 106 in the unit head 600 and the secondary channel 510 in the fluid appendage 500. When activated, the fluid pump 305 forces fluid through the internal channel 106 of the unit head 600 and into the secondary channel 510 of the fluid appendage 500. This pressurized delivery system ensures that the fluid is effectively delivered to the targeted area within the ear canal, facilitating the irrigation and suction process. The fluid pump 305 may be configured to be controlled by a controller. The controller may allow the user to activate or deactivate the fluid pump 305 as needed. In some embodiments, the fluid pump 305 may be connected to a controller that automates the fluid delivery process according to predefined schedules or in response to specific inputs from the user. In some embodiments, the fluid pump 305 may include a variable speed motor, allowing for adjustable fluid flow rates. This feature enables the user to customize the fluid delivery according to different irrigation needs. The fluid pump 305 may also incorporate a pressure regulator to ensure that the fluid is delivered at a consistent pressure.

Referring still to FIG. 2, the vacuum pump 304 is configured to create negative pressure for the removal of fluids and debris from the ear canal. The vacuum pump 304 may be housed within the gripping body 300 of the irrigation and suction device 100. The primary function of the vacuum pump 304 is to generate a negative pressure that facilitates the extraction of fluids and debris from the ear canal. The vacuum pump 304 is in fluid communication with the primary channel 506 of the fluid appendage 500 and the internal channels 108 of the unit head 600. When activated, the vacuum pump 304 creates a suction force that draws fluid and dislodged debris through the primary channel 506 of the fluid appendage 500, the internal channel 108 of the unit head 600, and into the repository 307 or other desired location. This process is important for maintaining a clear ear canal and preventing fluid buildup. The vacuum pump 304 is configured to be controlled by a controller. In some embodiments, the vacuum pump 304 may be connected to a controller that automates the suction process according to predefined schedules or in response to specific inputs from the user. In some embodiments, the vacuum pump 304 may include a variable speed motor, allowing for adjustable suction strength. This feature enables the user to customize the level of negative pressure applied, accommodating different levels of fluid and debris removal needs. The vacuum pump 304 may also incorporate a safety mechanism that prevents over-suction, protecting the ear canal from potential damage due to excessive negative pressure.

Referring now to FIGS. 3-6, depicted is a fluid appendage 500 for use in an ear irrigation and suction device such as, but not limited to, the irrigation and suction device 100 depicted in FIGS. 1-2. The fluid appendage 500 is configured to integrate seamlessly with the other components of the irrigation and suction device 100, including the unit head 600, fluid reservoir 306, and vacuum pump 304. The fluid appendage 500 is configured to facilitate the delivery and removal of fluids within the ear canal during the irrigation and suction process. The fluid appendage 500 may removably attach to the middle housing 400 and the unit head 600 of the irrigation and suction device 100, although other orientations of attachment between the fluid appendage 500 and the other components of the irrigation and suction device 100 are possible and encompassed within the scope of the present disclosure. The fluid appendage 500 is configured to be removably attached to the unit head 600 of the irrigation and suction device 100. This attachment may include a threaded connection, snap-fit, or bayonet mount, ensuring secure and reliable attachment during use. The fluid appendage 500 may optionally have a chamfered or beveled end, which may be used to facilitate insertion into the ear canal and to aid fluid flow dynamics.

Referring still to FIGS. 3-6, the fluid appendage 500 includes a primary channel 506 and a secondary channel 510, where the primary channel 506 extends from the proximal end 514 of the fluid appendage to a main tip 502 at the distal end 516 of the fluid appendage 500, and the secondary channel 510 extends from the proximal end 514 of the fluid appendage 500 to a nozzle 513 at the distal end 516 of the fluid appendage 500. The primary channel 506 of the fluid appendage 500 is configured to deliver a negative pressure supplied by the vacuum pump 304 to the ear canal. The primary channel 506 is configured to deliver a suction force that draws fluid and debris out of the ear canal, facilitating the removal process. The primary channel 506 defines a lumen, and may be referred to herein as the primary channel lumen 506. The main tip 502 of the fluid appendage 500, which is disposed at the distal end 516 of the fluid appendage 500, may optionally include a seal such as a rubber material to ensure a tight fit within the ear canal, preventing air leaks and enhancing suction efficiency. The secondary channel 510 is configured to supply fluid under positive pressure from the fluid reservoir 306 into the ear canal. The secondary channel 510 extends from a first side of the primary channel 506 at the proximal end 514 of the fluid appendage 500 to a second side of the primary channel 506 at the distal end 516 of the fluid appendage 500. Furthermore, in use, the secondary channel 510 protrudes further into the ear canal than the primary channel 506 because the nozzle 513 extends further outward than the main tip 502. This configuration allows for fluid delivery to the targeted area within the ear canal, aiding in the dislodging, dissolving, and expelling of debris.

Referring to FIG. 5, the fluid appendage 500 may include a plurality of appendage protrusions 527. The plurality of appendage protrusions 527 may be configured to mount the fluid appendage 500 to the unit head 600 or other components of the irrigation and suction device 100. The appendage protrusions 527 may snap fit or otherwise lock into the unit head 600 or other components of the device 100 so as to removably attach the fluid appendage 500 to the unit head 600 or other components of the device 100. In some embodiments, the plurality of appendage protrusions 527 are disposed between the primary channel 506 and the secondary channel 510. However, the location of the appendage protrusions 527 is not critical. Furthermore, the presence of the appendage protrusions 527 is not necessary, because the fluid appendage 500 may be removably attached to the unit head 600 or other component of the irrigation and suction device 100 through, for instance, a snap-fit connection with the first inlet 515 of the fluid appendage 500.

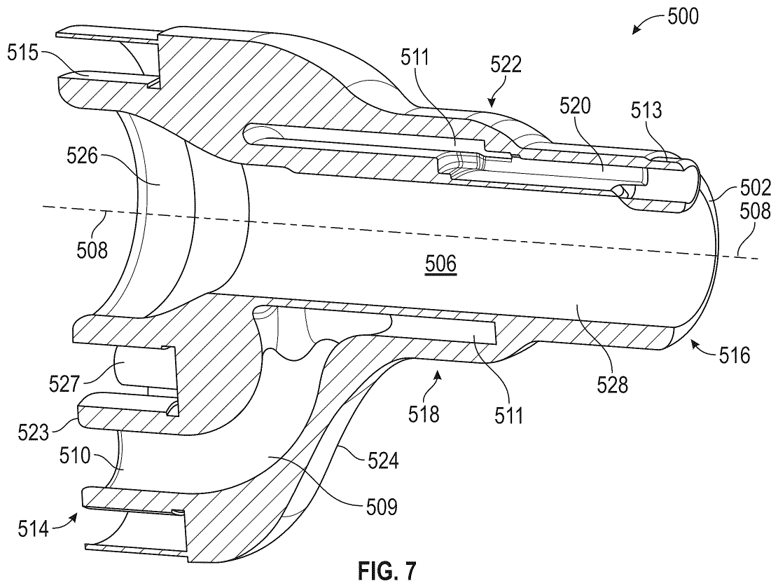

Referring now to FIG. 7, the secondary channel 510 within the fluid appendage 500 may include each of a dogleg lumen portion 509, a concentric lumen portion 511, and a top lumen portion 520 in fluid communication. The secondary channel 510 may wrap around a portion of the primary channel 506, so as to go from a first side of (e.g., beneath) the primary channel 506 at the proximal end 514 of the fluid appendage 500 to a second side of (e.g., above) the primary channel 506 at the distal end 516 of the fluid appendage 500. As seen in FIG. 7, the secondary channel 510 may wrap around the primary channel 506 such that the secondary channel 510 is on the second side of (e.g., above) the primary channel 506 in the middle portion 518 of the fluid appendage 500. In this example, the secondary channel 510 encircles the primary channel 506 in the middle portion 518, but not at either of the proximal end 514 of the fluid appendage 500 or the distal end 516 of the fluid appendage 500. Rather, at the proximal end 514, the secondary channel 510 is disposed within the fluid appendage 500 at a position removed from and below the primary channel 506, but not above the primary channel 506. In the middle portion 518, the secondary channel 510 encircles the primary channel 506. At the distal end 516, the secondary channel 510 is disposed directly adjacent to and above the primary channel 506 but not below the primary channel 506. The dogleg lumen portion 509 is disposed within a neck portion 524 of the fluid appendage 500 and includes a curve so as to direct fluid entering the secondary channel 510 at the proximal end 514 of the fluid appendage 500 into the concentric lumen portion 511. The secondary channel 510 is concentric with the primary channel 506 in the concentric lumen portion 511. The top lumen portion 520 is the section of the secondary channel 510 disposed in the top section 522 of the fluid appendage 500 but where the secondary channel 510 is no longer concentric with the primary channel 506, and is in fluid communication with both the concentric lumen portion 511 and the nozzle 513. Notably, the dogleg lumen portion 509 and the top lumen portion 520 are on opposite sides of the primary channel longitudinal axis 508.

Referring still to FIG. 7, in the configuration in which the secondary channel 510 wraps around the portion of the channel 506, the secondary channel 510 may form a concentric lumen portion 511 which is concentric with the primary channel 506. A fluid may be introduced to the secondary channel 510 through the second inlet 523 at the proximal end 514 of the fluid appendage 500, and fed into the ear canal by passing through the dogleg portion 509 in the neck portion 524 of the fluid appendage 500, the concentric lumen portion 511 in the middle portion 518 of the fluid appendage 500, the top lumen portion 520 in the top section 522 of the fluid appendage 500, and out through the nozzle 513 at the distal end 516 of the fluid appendage 500. As shown in FIG. 7, the secondary channel 510 feeds the concentric lumen portion 511 from the proximal end 514 below the primary channel 506, yet disperses the fluid from the nozzle 13 at the distal end 516 above the primary channel 506. In some embodiments, the dogleg portion 509, the concentric lumen portion 511, and the top lumen portion 520 have a sufficient volume so as to ensure that the fluid pressure remains consistent throughout the secondary channel 510.

As noted, the irrigation and suction device 100 may include a nozzle 513 at the distal end 516 of the fluid appendage 500. The nozzle 513 is configured to disperse fluid into the ear canal from the secondary channel 510. As such, the nozzle 513 is in fluid communication with the secondary channel 510. Thus, as depicted in FIG. 7, the nozzle 513 is in fluid communication with each of the dogleg lumen portion 509, concentric lumen portion 511, and top lumen portion 520 of the secondary channel 510. As seen in FIGS. 3, 4, and 7, the nozzle 513 may extend further distally than the main tip 502 of the fluid appendage 500. Thus, the nozzle 513 may extend further into the ear canal than the main tip 502 (and therefore the primary channel 506) when being used to irrigate and suction cerumen.

Referring now to FIG. 5, at the proximal end 514 of the fluid appendage 500, a first inlet 515 provides access to the primary channel 506 within the fluid appendage 500, and a second inlet 523 provides access to the secondary channel 510 within the fluid appendage 500. The first inlet 515 has a diameter which is greater than a diameter of the primary channel 506 at the main tip 502. As best seen in FIG. 7, the primary channel 506 may include a wider portion 526 within the first inlet 516, at the proximal end 514 of the fluid appendage 500, and a narrower portion 528 at the distal end 516 of the fluid appendage 500. The first inlet 515 may also have a greater diameter than the second inlet 523. This is advantageous for removing debris through the primary channel 506, and for providing a fluid with a positive pressure through the secondary channel 510.

When the fluid appendage 500 is connected to the unit head 600 or other components of the device 100, a negative pressure may be applied to the primary channel 506 through the first inlet 515 from the vacuum pump 304, and a fluid from the fluid reservoir 306 may be delivered into the secondary channel 510 through the second inlet 523. Tubing or hoses may be used to connect the fluid reservoir 306 to the second inlet 513, or a direct connection between the fluid reservoir 306 and the second inlet 513 can be made without tubing or hoses. Similarly, the vacuum pump 304 may be connected to the primary channel 506 with tubing or hoses, or may alternatively be directly connected to the primary channel 506 without tubing or hoses.

While the nozzle 513 may be a distal continuation of the secondary channel 510 beyond the main tip 205, the nozzle 513 may vary in diameter relative to other portions of the secondary channel 510. As shown in FIG. 7, the secondary channel 510 in one or more of the dogleg lumen portion 509, the concentric lumen portion 511, and the top lumen portion 520 has a diameter which is greater than the diameter of the secondary channel 510 in the nozzle 513. In such embodiments, the fluid pressure exiting the nozzle lumen 513 may be greater than the fluid pressure entering the secondary channel 510 at the proximal end 514 of the fluid appendage 500.

Referring now to FIG. 6, the nozzle 513 may include a non-circular opening 521. The perimeter of the opening 521 may include a flat portion 517 and a curved portion 519. In some embodiments, the nozzle perimeter curved portion 519 may be closer to the primary channel axis 508 than the nozzle perimeter flat portion 517. In other words, the nozzle perimeter flat portion 519 is disposed further radially than the nozzle perimeter curved portion 519 with respect to the primary channel axis 508. In this configuration, the nozzle opening 521 may be used to create a predetermined spray pattern of the fluid exiting the nozzle 513 from the secondary channel 510.

In some embodiments, the fluid appendage 500 is designed to fit better in the ear of a dog specifically, and includes a longer length from the proximal end 514 to the distal end 516, and also a curved main tip 502 and/or curved nozzle 513. The internal features of the fluid appendage 500 may otherwise remain the same. However, these modifications are not strictly necessary when the fluid appendage 500 is used with the irrigation and suction device 100 to irrigate and suction the ear of a dog, and uses of the fluid appendage 500 without one or both of these modifications with the irrigation and suction device 100 to irrigate and suction the ear of a dog are encompassed within the scope of the present disclosure.

The modular design of the irrigation and suction device 100 allows for the attachment of various accessory components, or alternative fluid appendages, providing versatility in the device's application. This modularity enables the user to customize the irrigation and suction process according to different needs and preferences.

Some embodiments of the irrigation and suction device 100 may include a controller. The controller may be used in operation of both the fluid reservoir 306 and the vacuum pump 304. The controller may be an electronic unit integrated within the gripping body 300 of the irrigation and suction device 100. The controller is configured to manage the operation of the fluid pump 305 and the vacuum pump 304 based on user inputs and predefined schedules. The controller may include a microprocessor, memory, and various input/output interfaces to handle the control logic and user interactions. The control system includes a user interface. The user interface may include buttons or switches which may be disposed on the gripping body 300. The user interface may allow the user to activate or deactivate the pumps 304, 305 within the irrigation and suction device 100. Activation and deactivation of the pumps 304, 305 within the irrigation and suction device 100 effectively control operation of the irrigation and suction device 100. The user interface may also include indicator lights or a display screen to provide real-time feedback on the device's status, such as power levels, fluid levels, and operational modes. The controller is configured to actuate the fluid pump 305 and the vacuum pump 304 between active and deactivated states. When a user presses the activation button for the fluid pump 305, the controller sends a signal to initiate the pump, causing the fluid pump 305 to generate positive pressure and deliver fluid from the reservoir to the ear canal through the secondary channel 510. Similarly, when the vacuum pump 304 activation button is pressed, the controller activates the vacuum pump 304 to create negative pressure, enabling the removal of fluid and debris from the ear canal via the primary channel 506. The controller may include programmable schedules to automate the operation of the pumps 304, 305. This feature allows the user to set specific times or intervals for the pumps 304, 305 to activate and deactivate. For example, the controller can be programmed to alternate between fluid delivery and suction cycles.

The control system may incorporate various sensors that provide feedback to the controller, ensuring optimal performance and safety. These sensors can include pressure sensors to monitor the pressure within the fluid channels, such that the fluid delivery and suction pressures remain within safe and effective ranges, fluid level sensors to detect the fluid level in the fluid reservoir 306, and vacuum sensors to monitor the negative pressure generated by the vacuum pump 304. The feedback from these sensors is processed by the controller, which adjusts the pump operations as necessary to maintain optimal irrigation and suction conditions. The controller may include safety features to prevent over-pressurization or over-suction. For example, the controller may be programmed to shut off the pumps 304, 305 if the pressure readings exceed predefined thresholds. Additionally, the system may include a manual override function, allowing the user to immediately stop the pumps 304, 305 if necessary. In some embodiments, the controller may be equipped with wireless communication capabilities, enabling remote control via a smartphone or other mobile devices. This feature may allow remote monitoring and adjustments.

The controller may also be configured to accommodate various power sources, including rechargeable batteries or direct power connections. As depicted, the irrigation and suction device 100 includes an internal battery 700. The irrigation and suction device 100 may include an internal battery 700 or may alternatively be powered through an external power source. In embodiments where the irrigation and suction device 100 includes a rechargeable internal battery 700, the controller may manage the charging process, ensuring that the battery 700 is properly charged and maintained.

The controller may be configured to use a plurality of operational modes. These modes are configured to address different cleaning needs and user preferences. The modes can be selected through the user interface on the gripping body 300. One of the plurality of operational modes may be manual mode. In manual mode, the user has direct control over the activation and deactivation of the fluid pump 305 and vacuum pump 304. This mode allows users to manually start and stop the irrigation and suction process as needed. The user can press the activation buttons to initiate the fluid delivery or suction and release them to stop the operation. Another of the operational modes is automatic mode. Automatic mode automates the fluid delivery and suction cycles. In automatic mode, the controller may be programmed to alternate between delivering fluid and applying suction at set intervals. This mode may provide a steady rhythm of fluid input and debris removal. Users may be able to set the duration and frequency of each cycle in automatic mode. Even another of the operational modes is pulsed mode. Pulsed mode may employ intermittent bursts of fluid delivery and suction. This mode is configured to enhance the dislodging of stubborn earwax and debris by applying fluid and suction in rapid, controlled pulses. The pulsing action can help break up compacted earwax. The controller can adjust the intensity and frequency of the pulses based on user settings or predefined schedules.

Another operational mode may be continuous mode. Continuous mode provides a steady and uninterrupted flow of fluid and suction. The continuous operation ensures that fluid is constantly supplied to the ear canal while simultaneously removing debris. Users can set the desired duration for continuous operation, and the controller will maintain the fluid and suction flow for the specified period. The controller may allow users to create custom schedules for fluid delivery and suction. Users may define specific times and durations for the pumps to activate and deactivate. For example, a user can set the device to operate in automatic mode for a few minutes, followed by a switch to pulsed mode for intensive cleaning. These schedules can be saved and reused, providing a personalized cleaning regimen. The operational modes can include adjustable intensity settings for both fluid delivery and suction. Users can select low, medium, or high intensity levels based on their comfort and cleaning requirements. The controller adjusts the pump speeds accordingly, ensuring that the fluid pressure and suction strength match the selected intensity.

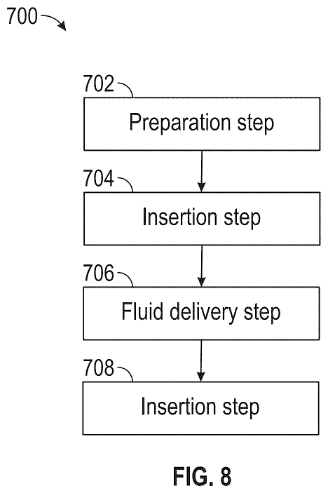

FIG. 8 illustrates a first flowchart of a non-limiting example irrigation and suction method 700. The irrigation and suction method 700 begins with a preparation step 702 in which a user selects the desired operational mode. The preparation step 702 may include filling the fluid reservoir 306 with an ear irrigation solution. Next, in the insertion step 704, the user gently inserts the fluid appendage into an ear canal of a subject. In this step, the user ensures that the nozzle 513 (housing the secondary channel 510) is inserted into the ear canal deeper than the main tip 502 (housing the primary channel 506). In the fluid delivery step 706, the user activates the fluid pump 305 using the user interface. In the suction application step 708, the user activates the vacuum pump 304 using the user interface. The fluid delivery step 706 and the suction application step 708 may be performed simultaneously or sequentially for the efficient removal of ear wax from the ear canal of the subject. The subject may be a human or animal such as, but not limited to, a dog or cat.

Referring now to FIG. 9, depicted is an animal ear fluid appendage 800 for use in an ear irrigation and suction device such as, but not limited to, the irrigation and suction device 100 depicted in FIGS. 1-2. The animal ear fluid appendage 800 is configured to integrate seamlessly with the other components of the irrigation and suction device 100, including the unit head 600, fluid reservoir 306, and vacuum pump 304. The animal ear fluid appendage 800 is configured to facilitate the delivery and removal of fluids within the ear canal of an animal, for example a dog, during the irrigation and suction process. The animal ear fluid appendage 800 may be removably attached to the middle housing 400 and the unit head 600 of the irrigation and suction device 100, although other orientations of attachment between the animal ear fluid appendage 800 and the other components of the irrigation and suction device 100 are possible and encompassed within the scope of the present disclosure. The animal ear fluid appendage 800 is configured to be removably attached to the unit head 600 of the irrigation and suction device 100. This attachment may include a threaded connection, snap-fit, or bayonet mount, ensuring secure and reliable attachment during use.

Referring still to FIG. 9, the animal ear fluid appendage 800 includes a primary channel 806 and a secondary channel 810, where the primary channel 806 extends from a proximal end 814 of the animal ear fluid appendage 800 to a main tip 802, and the secondary channel 810 extends from the proximal end 814 of the animal ear fluid appendage 800 to a nozzle (not depicted in FIG. 9 but structurally the same as the nozzle 513), adjacent to the main tip 902. Although the animal ear fluid appendage 800 is described as having the nozzle 513, in alternative embodiments, there is no nozzle. The primary channel 806 of the animal ear fluid appendage 800 is configured to deliver a negative pressure supplied by the vacuum pump 304 to the ear canal of an animal. The primary channel 806 is configured to deliver a suction force that draws fluid and debris out of the ear canal, facilitating the removal process. The primary channel 806 defines a lumen, and may be referred to herein as the primary channel lumen 806. The main tip 802 of the animal ear fluid appendage 800, which is disposed distally relative to the proximal end 816 of the animal ear fluid appendage 800, may optionally include a seal such as a rubber material to ensure a tight fit within a proximal end 852 of an elongate tubular member 850 that is attached to the main tip 802 of the animal ear fluid appendage 800. The secondary channel 810 is configured to supply fluid under positive pressure from the fluid reservoir 306 into the ear canal of an animal. The secondary channel 810 extends from a first side of the primary channel 806 to the proximal end 814 of the animal ear fluid appendage 800. The nozzle may be a distal continuation of the secondary channel 810 beyond the main tip 802, and the nozzle may vary in diameter relative to other portions of the secondary channel. The nozzle may further include a non-circular opening similar to the one illustrated in FIG. 6.

Referring still to FIG. 9, the animal ear fluid appendage 800 may include a plurality of appendage protrusions, which are not illustrated in FIG. 9 but are structurally the same as the appendage protrusions 527. The plurality of appendage protrusions may be configured to mount the animal ear fluid appendage 800 to the head unit 600 or other components of the irrigation and suction device 100. The appendage protrusions may snap fit or otherwise lock into the unit head 600 or other components of the device 100 so as to removably attach the animal ear fluid appendage 800 to the unit head 600 or other components of the device 100. In some embodiments, the appendage protrusions are disposed between the primary channel 806 and the secondary channel 810. However, the location of the appendage protrusions is not critical. Furthermore, the presence of the appendage protrusions is not necessary, because the animal ear fluid appendage 800 may be removably attached to the unit head 600 or other component of the irrigation and suction device 100 through, for instance, a snap-fit connection with the first inlet 815 of the animal ear fluid appendage 800.

Referring still to FIG. 9, at the proximal end 814 of the animal ear fluid appendage 800, a first inlet 815 provides access to the primary channel 806 within the animal ear fluid appendage 800, and a second inlet 823 provides access to the secondary channel 810 within the animal ear fluid appendage 800. The first inlet 815 has a diameter which is greater than the diameter of the primary channel 806 at the main tip 802. The primary channel 806 may include a wider portion within the first inlet 816, at the proximal end 814 of the animal ear fluid appendage 800, and a narrower portion at the main tip 802 of the animal ear fluid appendage 800. The first inlet 815 may also have a greater diameter than the second inlet 823. This is advantageous for removing debris through the primary channel 806, and for providing a fluid with a positive pressure through the secondary channel 810.

When the animal ear fluid appendage 800 is connected to the unit head 600 or other components of the device 100, a negative pressure may be applied to the primary channel 806 through the first inlet 815 from the vacuum pump 304, and a fluid from the fluid reservoir 306 may be delivered into the secondary channel 810 through the second inlet 823. Tubing or hoses may be used to connect the fluid reservoir 306 to the second inlet 813, or a direct connection between the fluid reservoir 306 and the second inlet 813 can be made without tubing or hoses. Similarly, the vacuum pump 304 may be connected to the primary channel 806 with tubing or hoses, or may alternatively be directly connected to the primary channel 806 without tubing or hoses.

The animal ear fluid appendage 800 further includes the elongate tubular member 850 that has an elongate tubular member proximal end 852, an elongate tubular member distal end 854, an elongate tubular member intermediate portion 856, an elongate tubular member flange 858, and an elongate tubular member nozzle 860. The elongate tubular member proximal end 852 extends over the main tip 802 of the animal ear fluid appendage 800. The elongate tubular member proximal end 852 narrows in a direction toward the elongate tubular member distal end 854. In other words, the elongate tubular member proximal end 852 includes a portion which fits over the main tip 802 and which has a larger circumference than the rest of the elongate tubular member 850. The elongate tubular member intermediate portion 856 extends between the elongate tubular member proximal end 852 and the elongate tubular member distal end 854. The elongate tubular member intermediate portion 856 has a bend 862 that is at an angle of greater than 90 degrees (relative to the longitudinal axis of the tubular member proximal end 852) and is configured to allow the elongate tubular member distal end 854 to access the ear canal of an animal more easily. Although the bend 862 is illustrated as having angle that is greater than 90 degrees, in alternative embodiments, the angle can be any suitable angle including, but not limited to, 80 degrees, 90 degrees, 100 degrees, 120 degrees, or 130 degrees. The elongate tubular member distal end 854 is defined by the flange 858, which has a circumference that is larger than the circumference of the elongate tubular member intermediate portion 856. The elongate tubular member nozzle 860 is an extension of the nozzle described above, and can be structurally the same as the nozzle described in relation to the fluid appendage 500 described above. Therefore, the elongate tubular member nozzle 860 is an extension of the secondary channel 810. The elongate tubular member nozzle 860 may extend longitudinally beyond the elongate tubular member distal end 854. The elongate tubular member 850 also defines an elongate tubular member lumen 864. The elongate tubular member lumen 864 is an extension of the first channel 806 and extends from the elongate tubular member proximal end 852 to an opening located at the elongate tubular member distal end 854. The elongate tubular member lumen 864 may be concentric with the secondary channel 810 from the elongate tubular member proximal end 852 to the elongate tubular member distal end 854. However, this is not strictly necessary. The elongate tubular member 850 may be made from a rubber material; however, the elongate tubular member 850 can be made from any suitable material. The elongate tubular member 850 being rubber is beneficial at least because it allows the elongate tubular member 850 to bend while accessing the ear canal of an animal.

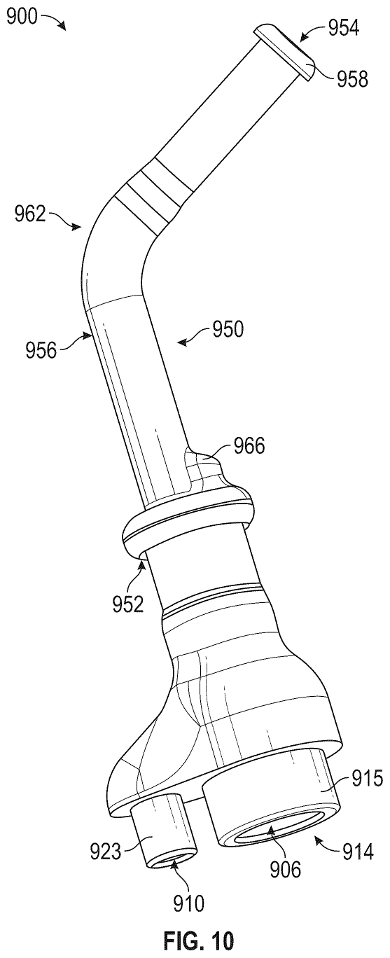

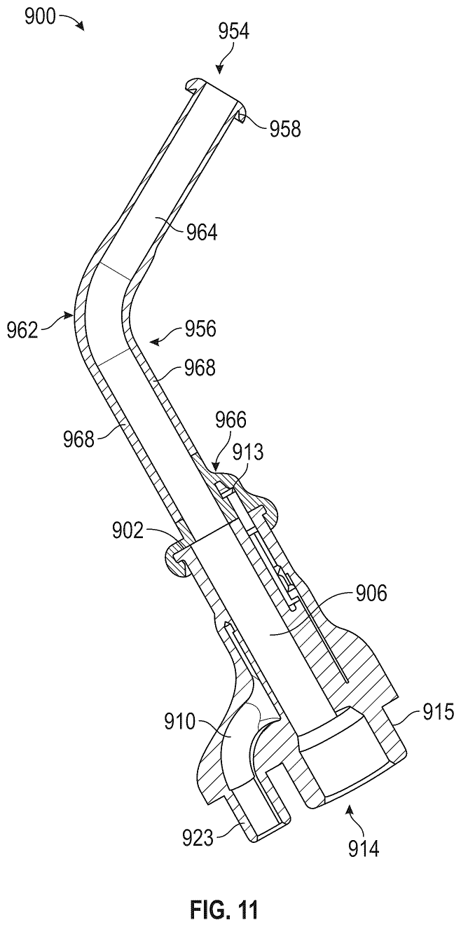

Referring now to FIGS. 10-13, depicted is another example animal ear fluid appendage 900 for use in an ear irrigation and suction device such as, but not limited to, the irrigation and suction device 100 depicted in FIGS. 1-2. The animal ear fluid appendage 900 is configured to integrate seamlessly with the other components of the irrigation and suction device 100, including the unit head 600, fluid reservoir 306, and vacuum pump 304. The animal ear fluid appendage 900 is configured to facilitate the delivery and removal of fluids within the ear canal of an animal, for example a dog, during the irrigation and suction process. The animal ear fluid appendage 900 may be removably attached to the middle housing 400 and the unit head 600 of the irrigation and suction device 100, although other orientations of attachment between the animal ear fluid appendage 900 and the other components of the irrigation and suction device 100 are possible and encompassed within the scope of the present disclosure. The animal ear fluid appendage 900 is configured to be removably attached to the unit head 600 of the irrigation and suction device 100. This attachment may include a threaded connection, snap-fit, or bayonet mount, ensuring secure and reliable attachment during use.

Referring still to FIGS. 10-13, the animal ear fluid appendage 900 includes a primary channel 906 and a secondary channel 910, where the primary channel 906 extends from a proximal end 914 of the animal ear fluid appendage 900 to a main tip 902, and the secondary channel 910 extends from the proximal end 914 of the animal ear fluid appendage 900 to a nozzle 913 which is adjacent to the main tip 902. Although the animal ear fluid appendage 900 is described as having the nozzle 913, in alternative embodiments, other configurations are possible and encompassed within the scope of the present disclosure. The nozzle 913 serves to fluidly connect the secondary channel 910 within the proximal end 914 to the elongate tubular member perimeter channel 968 within the elongate tubular member intermediate portion 956. The nozzle may be housed within the elongate tubular member protrusion 966. The secondary channel 910 is configured to supply fluid under positive pressure from the fluid reservoir 306 into the ear canal of an animal. The secondary channel 910 extends from a first side of the primary channel 906 to the proximal end 914 of the animal ear fluid appendage 900. The nozzle 913 is a distal continuation of the secondary channel 910 beyond the main tip 902, and the nozzle 913 may vary in diameter relative to other portions of the secondary channel 910.

The primary channel 906 of the animal ear fluid appendage 900 is configured to deliver a negative pressure supplied by the vacuum pump 304 to the ear canal of an animal. The primary channel 906 is configured to deliver a suction force that draws fluid and debris out of the ear canal, facilitating the removal process. The primary channel 906 defines a lumen, and may be referred to herein as the primary channel lumen 906. The main tip 902 of the animal ear fluid appendage 900, which is disposed distally relative to the proximal end 914, may optionally include a seal such as a rubber material to ensure a tight fit within a proximal end 952 of the elongate tubular member 950 which is attached to the main tip 902 of the animal ear fluid appendage 900.

Referring still to FIGS. 10-13, the animal ear fluid appendage 900 may include a plurality of appendage protrusions, which are not illustrated in FIGS. 10-13 but which are structurally the same as the appendage protrusions 527. The plurality of appendage protrusions may be configured to mount the animal ear fluid appendage 900 to the head unit 600 or other components of the irrigation and suction device 100. The appendage protrusions may snap fit or otherwise lock into the unit head 600 or other components of the device 100 so as to removably attach the animal ear fluid appendage 900 to the unit head 600 or other components of the device 100. In some embodiments, the appendage protrusions are disposed between the primary channel 906 and the secondary channel 910. However, the location of the appendage protrusions is not critical. Furthermore, the presence of the appendage protrusions is not necessary, because the animal ear fluid appendage 900 may be removably attached to the unit head 600 or other component of the irrigation and suction device 100 through, for instance, a snap-fit connection with the first inlet 915 of the animal ear fluid appendage 900.

Referring still to FIGS. 10-13, at the proximal end 914 of the animal ear fluid appendage 900, a first inlet 915 provides access to the primary channel 906 within the animal ear fluid appendage 900, and a second inlet 923 provides access to the secondary channel 910 within the animal ear fluid appendage 900. The first inlet 915 has a diameter which is greater than the diameter of the primary channel 906 at the main tip 902. The primary channel 906 may include a wider portion within the first inlet 916, at the proximal end 914 of the animal ear fluid appendage 900, and a narrower portion at the main tip 902 of the animal ear fluid appendage 900. The first inlet 915 may also have a greater diameter than the second inlet 923. This is advantageous for removing debris through the primary channel 906, and for providing a fluid with a positive pressure through the secondary channel 910.

Referring still to FIGS. 10-13, when the animal ear fluid appendage 900 is connected to the unit head 600 or other components of the device 100, a negative pressure may be applied to the primary channel 906 through the first inlet 915 from the vacuum pump 304, and a fluid from the fluid reservoir 306 may be delivered into the secondary channel 910 through the second inlet 923. Tubing or hoses may be used to connect the fluid reservoir 306 to the second inlet 923, or a direct connection between the fluid reservoir 306 and the second inlet 923 can be made without tubing or hoses. Similarly, the vacuum pump 304 may be connected to the primary channel 906 with tubing or hoses, or may alternatively be directly connected to the primary channel 906 without tubing or hoses.

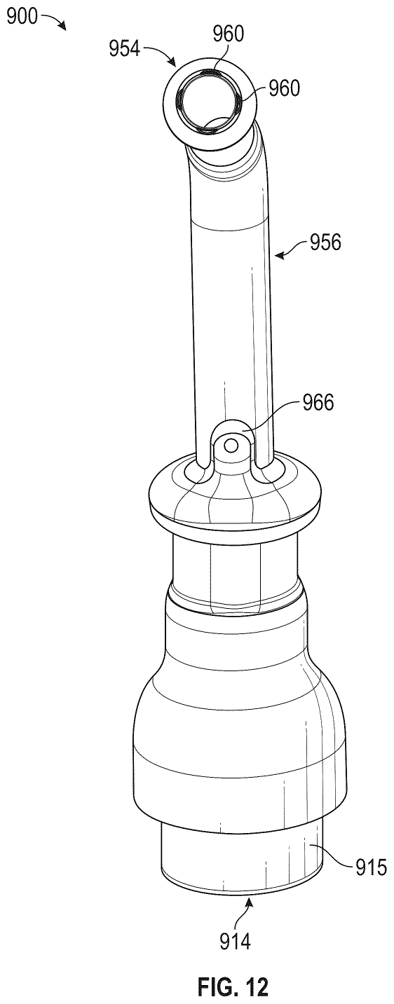

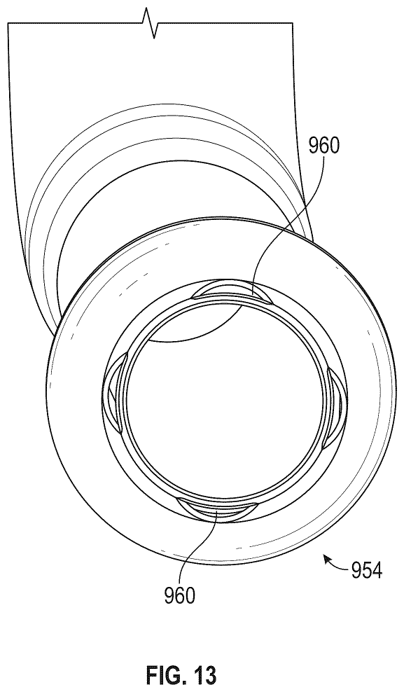

Referring still to FIGS. 10-13, the animal ear fluid appendage 900 further includes the elongate tubular member 950 having an elongate tubular member proximal end 952, an elongate tubular member distal end 954, an elongate tubular member intermediate portion 956, an elongate tubular member flange 958, and a plurality of elongate tubular member nozzles 960. The elongate tubular member proximal end 952 extends over the main tip 902 of the animal ear fluid appendage 900. In the illustrated embodiment, the elongate tubular member proximal end 952 is connected to the main tip 902 by a boot lock; however, any suitable configuration can be used to secure the elongate tubular member 950 to the main tip 902. The elongate tubular member proximal end 952 narrows in a direction toward the elongate tubular member distal end 954. The elongate tubular member intermediate portion 956 extends between the elongate tubular member proximal end 952 and the elongate tubular member distal end 954. The elongate tubular member intermediate portion 956 has a bend 962 which is at an angle of greater than 90 degrees (relative to the elongate tubular member proximal end 952) and is configured to allow the elongate tubular member distal end 954 to access the ear canal of an animal more easily. Although the bend 962 is illustrated as having angle that is greater than 90 degrees, in alternative embodiments, the angle can be any suitable angle including, but not limited to, 80 degrees, 90 degrees, 100 degrees, 120 degrees, or 130 degrees. The elongate tubular member distal end 954 is defined by the flange 958 that has a circumference which is larger than the circumference of the elongate tubular member intermediate portion 956. The plurality of elongate tubular member nozzles 960 (best seen in FIGS. 12-13) are defined by the flange 958, and can be an extension of the nozzle described above. In the illustrated embodiment the plurality of elongate tubular member nozzles 960 are crescent shaped; however, any suitable shape can be used. The plurality of tubular member nozzles 960 being crescent shaped is beneficial at least because this can create a positive pressure. The plurality of elongate tubular member nozzles 960 are extensions of the secondary channel 910.

Referring still to FIGS. 10-13, the elongate tubular member 950 also defines an elongate tubular member lumen 964. The elongate tubular member lumen 964 is an extension of the first channel 906 and extends from the elongate tubular member proximal end 952 to an opening defined by the elongate tubular member distal end 954. The elongate tubular member 950 is made from a rubber material; however, the elongate tubular member 950 can be made from any suitable material. The elongate tubular member 950 being rubber is beneficial at least because it allows the elongate tubular member 950 to bend while accessing the ear canal of an animal. The elongate tubular member 950 further includes an elongate tubular member protrusion 966 and the elongate tubular member perimeter channel 968 which extends along the inner circumference of the elongate tubular member 950 from the elongate tubular member protrusion 966 to the elongate tubular member distal end 954. The elongate tubular member perimeter channel 968 is in fluid communication with the nozzle 913, and is an extension of the secondary channel 910. The elongate tubular member protrusion 966 serves to allow the elongate tubular member perimeter channel 968 to be in fluid communication with the secondary channel 910. Further, the plurality of elongate tubular member nozzles 960 are in fluid communication with the elongate tubular member perimeter channel 968.

All of the components of the animal ear fluid appendages 800, 900 may be constructed from materials suitable for medical devices such as, but not limited to, medical-grade plastics, stainless steel, acrylonitrile butadiene styrene (ABS) filament, thermoplastic polyurethane (TPU) filament, or any other suitable materials. In some embodiments, the animal ear fluid appendage 800, 900 is constructed such that certain parts are composed of ABS and certain parts are composed of TPU.

Certain embodiments of the devices and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the devices and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. An irrigation and suction device for an ear comprising:
a fluid reservoir;
a vacuum pump; and
a fluid appendage having a primary channel therein and a secondary channel therein, wherein the primary channel is in fluid communication with the vacuum pump and the secondary channel is in fluid communication with the fluid reservoir, the fluid appendage further comprising a main tip where the primary channel terminates and a nozzle where the secondary channel terminates, wherein the nozzle extends longitudinally outward beyond the main tip.

2. The irrigation and suction device of claim 1, wherein the secondary channel includes a dogleg lumen portion, a concentric lumen portion, and a top lumen portion.

3. The irrigation and suction device of claim 2, wherein the primary channel longitudinally defines a primary channel axis, the dogleg lumen portion is on a first side of the longitudinal axis, and the top lumen portion is on a second side of the primary channel axis.

4. The irrigation and suction device of claim 2, wherein the concentric lumen portion is concentric with the primary channel in a middle portion of the fluid appendage.

5. The irrigation and suction device of claim 1, wherein the nozzle includes a non-circular opening.

6. The irrigation and suction device of claim 5, wherein the nozzle has a perimeter which includes a flat portion and a curved portion, where the curved portion is closer to the primary channel axis than the flat portion is.

7. The irrigation and suction device of claim 1, wherein the main tip is curved.

8. An irrigation and suction device for an ear comprising:
a fluid reservoir;
a vacuum pump; and
an animal ear fluid appendage having a primary channel therein and a secondary channel therein, wherein the primary channel is in fluid communication with the vacuum pump and the secondary channel is in fluid communication with the fluid reservoir, the animal ear fluid appendage having an elongate tubular member extending from an elongate tubular member proximal end at a main tip to an elongate tubular member distal end, wherein the elongate tubular member has a bend in an elongate tubular member intermediate portion.

9. The irrigation and suction device of claim 8, wherein the elongate tubular member distal end defines a flange, the flange having a larger circumference than the elongate tubular member intermediate portion.

10. The irrigation and suction device of claim 9, wherein the flange defines a plurality of crescent-shaped nozzles in fluid communication with the secondary channel.

11. The irrigation and suction device of claim 8, wherein the elongate tubular member proximal end fits over the main tip.

12. The irrigation and suction device of claim 8, wherein the secondary channel terminates at a nozzle, wherein the nozzle extends longitudinally outward beyond the elongated tubular member distal end.

13. The irrigation and suction device of claim 8, further comprising a seal between the main tip and the elongate tubular member proximal end.

14. The irrigation and suction device of claim 8, wherein the bend is at an angle of greater than 90 degrees relative to a longitudinal axis of the elongate tubular member proximal end.

15. A fluid appendage for an irrigation and suction device comprising:
a main tip comprising a first inlet providing access to a primary channel and a second inlet providing access to a secondary channel;
an elongate tubular member extending from an elongate tubular member proximal end at the main tip to an elongate tubular member distal end with an elongate tubular member intermediate portion therebetween, wherein the elongate tubular member is connected to the main tip; and
a bend in the elongate tubular member intermediate portion;
wherein the elongate tubular member is configured to allow passage of a negative pressure through the primary channel to the elongate tubular member distal end, and to allow passage of a fluid through the secondary channel to the elongate tubular member distal end; and
wherein the primary channel is concentric with the secondary channel in the elongate tubular member intermediate portion.

16. The fluid appendage of claim 15, wherein the secondary channel terminates at a nozzle on the distal end, the nozzle extending longitudinally beyond the distal end.

17. The fluid appendage of claim 15, further comprising an elongate tubular member protrusion housing a nozzle which fluidly connects the secondary channel to an elongate tubular member perimeter channel within the elongate tubular member intermediate portion.

18. The fluid appendage of claim 15, the primary channel is concentric with the secondary channel from the elongate tubular member proximal end to the elongate tubular member distal end.

19. The fluid appendage of claim 15, wherein the bend is at an angle of greater than 90 degrees relative to a longitudinal axis of the elongate tubular member proximal end.

20. A method of irrigating and suctioning an ear, the method comprising:
inserting the main tip and the nozzle of the fluid appendage of the irrigation and suction device of claim 1 into an ear canal of a subject;
applying negative pressure to the ear canal through the primary channel in the fluid appendage; and
pumping a fluid from the fluid reservoir into the ear canal through the secondary channel in the fluid appendage;
wherein the nozzle extends farther into the ear canal than the main tip.
